(19) United States
(12) Patent Application Publication
(10) Pub. No.: US 2021/0016036 A1
(43) Pub. Date: Jan. 21, 2021

(54) WEARABLE SUCTION ASPIRATOR FOR NASAL, ORAL AND TRACHEOSTOMY AIRWAY SECRETIONS

(71) Applicant: Neotech Products LLC, Valencia, CA (US)

(72) Inventors: Jon S. Guy, Encinitas, CA (US); Marlo Rosbrook, San Diego, CA (US); Craig McCrary, Valencia, CA (US)

(73) Assignee: Neotech Products LLC, Latrobe, PA (US)

(21) Appl. No.: 16/916,531

(22) Filed: Jun. 30, 2020

Related U.S. Application Data
(60) Provisional application No. 62/874,579, filed on Jul. 16, 2019.

(51) Int. Cl. A61M 16/04; A61M 16/06

(52) U.S. Cl. CPC A61M 16/0463; A61M 16/0488; A61M 16/0418; A61M 2210/0625; A61M 16/0666; A61M 16/0484; A61M 2210/0618; A61M 16/0427

(57) ABSTRACT

A wearable suction catheter that can be used without clinician control. The suction catheter attaches on or near the patient with a patient interface configured for the shape of the nose, mouth or tracheostomy tube. The patient interface includes at least two radial duct openings and each end of the interface is connected to airway tubing ending in a wye. The wye end of the catheter includes a cleaning access port and is attached to a suction source that provides continuous or intermittent suction.

### RELATED APPLICATION

This application claims the benefit of U.S. Provisional Application No. 62/874,579, filed on Jul. 16, 2019.

### BACKGROUND OF THE INVENTION

The present invention relates to suction devices for medical purposes and in particular to wearable suction aspirators used for removing airway secretions in and around the nose and mouth or around tracheostomy tubes of patients. Patients with excessive saliva production, intubated patients and patients with reduced motor functions express uncontrolled secretions in and around nasal, oral, and tracheostomy openings.

Current institutional practice is for clinicians to use a hand held suction catheter to remove secretions from patients not able to manage their own secretions. Suction catheters consist of a wand with a tip opening at the proximal end near the patient and the other end is attached to a circuit connected to a suctioning system. The clinician holds the suction catheter and directs the tip to remove the secretions in and around the patient's airways. After suctioning, the catheter wand is typically placed in an open container or on the patient's bed for use later on the same patient. Clinicians will normally attend to a patient every 2 to 4 hours. Used suction catheters are routinely disposed of and replaced once per day.

Current home care practice is for caregivers to use a suction catheter attached to a portable suctioning source and wrap a towel or bandage around the areas getting wet, soaking up the secretions.

There are many inherent problems with the current suctioning practices. Patient suctioning is often deferred when the clinician is called to attend to an emergency, or other duties. When a patient is not suctioned secretions build up on the skin and dressings, causing damage from the prolonged wetness. This leads to secondary problems such as bacteria growth and infections.

Excessive secretions put intubated patients at risk for ventilator acquired pneumonia (VAP) if secretions are aspirated by the patient. Ventilator acquired pneumonia increases the length of patient stay on a ventilator, increases patient health risks and increases financial liability for the hospital.

Reusing suction catheters leads to increased bacterial growth opportunities to the patient and clinicians, increasing hospital costs.

There is a significant need to address the management of excessive patient respiratory secretions. Current devices are not effective in managing the issue, leading to an increase in bacterial infections, care giver time and hospital costs.

Accordingly, there is a need for a device or devices to assist in organizing the tubes and cables around a patient in a medical treatment bed. Thus, it would be advantageous to have a device that can lift and hold the tubes and cables in a treatment bed so as to organize the same. The present invention fulfills these needs and provides other related advantages.

### SUMMARY OF THE INVENTION

The present invention comprises a wearable suction catheter that can remove airway secretions in and near the nose and mouth, and around the tracheostomy tube without clinician control. The invention suction catheter does not require bedside storage reducing the chance of cross-contamination. The invention reduces the potential for ventilator acquired pneumonia by continually removing saliva build-up. In one embodiment the present invention is a suction catheter that attaches to the patient with an interface configured for the shape of the mouth. The patient interface includes at least two radial duct openings. Each end of the interface is connected to tubing ending in a wye conduit. The wye conduit end of the catheter includes a cleaning access port and is attached to a suction source that provides continuous or intermittent suction.

The wearable suction catheter includes: (a) a rigid plastic tubular first duct configured to directly endwise connect to a suction circuit providing a negative pressure source; (b) rigid plastic tubular second ducts integral with the said first duct and diverging endwise therefrom with a wye configuration to receive negative pressure from said suction circuit; (c) relatively flexible plastic tubing connected in series to end portion of the said second ducts to extend therefrom providing a relative length of said flexible tubing to be placed on the patient; (d) a relatively rigid plastic third duct connected to the said flexible plastic tubing ending with two or more radial openings along the length of the said third duct with or without: (i) a relatively flexible plastic forth duct extending radial openings from the third duct extending into the nose or mouth; or (ii) a shaped third duct that is placed proximal to the trachea tube.

The wearable suction catheter may include a rigid plastic fifth duct integral with the said first duct diverging perpendicular to the first duct to receive a flow of water or saline.

The present invention is directed to a wearable suction catheter having a plastic patient aspirator interface having a tubular body and at least two aspiration openings, along with a first airway tube and a second airway tube, both connected endwise to opposite ends of the tubular body. The other ends of the airway tubes are connected to a wye conduit having a conduit inlet and first and second conduit branches. The first conduit branch is connected to the first airway tube and the second conduit branch is connected to the second airway tube, and the conduit inlet is configured for connection to a vacuum source.

In one preferred embodiment, the tubular body is configured for attachment to a patient's mouth such that the at least two aspiration openings are disposed proximate to either side of the patient's mouth. In this embodiment, the patient aspirator interface may further have at least one oral aspiration tube configured for insertion into one side of the patient's mouth, ore preferably, two oral aspiration tubes configured for insertion into both sides of the patient's mouth.

In another preferred embodiment, the tubular body is configured for attachment to a patient's nares such that the at least two aspiration openings are disposed proximate to either side of the patient's nares. In this embodiment, the patient aspirator interface may further have two nasal aspiration tubes configured for insertion into the patient's nares.

In another preferred embodiment, the tubular body is shaped as a torus and configured for disposal around a patient's tracheostomy tube and has a latitudinal curvature configured to match a patient's neck proximate to the tracheostomy tube, such that the aspiration openings are disposed proximate to the tracheostomy tube. In this embodiment, the patient aspirator interface preferably has four aspiration openings.

The wye conduit may also have a cleaning port disposed between the conduit inlet and the conduit branches. A cap may be provided on the cleaning port, configured for selectively opening or closing the cleaning port.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

Some embodiments of the present invention are illustrated as an example and are not limited by the figures of the accompany drawings, in which like references may indicate similar elements and in which:

The accompanying drawings illustrate the invention. In such drawings:

FIG. 1 depicts a system diagram of a wearable suction catheter;

FIG. 2 illustrates a wye conduit;

FIG. 3 shows a patient interface for the mouth;

FIG. 4 shows a patient interface for the nose;

FIG. 5 shows a patient interface for a trachea;

FIG. 5a shows a side view of the patient interface for a trachea;

FIG. 6 illustrates one embodiment using the wearable suction catheter for mouth secretions;

FIG. 7 illustrates another embodiment using the wearable suction catheter for the nose secretions; and

FIG. 8 illustrates another embodiment using the wearable suction catheter for secretions around the tracheostomy tube.

### DETAILED DESCRIPTION OF THE INVENTION

As shown in the drawings, a wearable aspiration device for use in medical settings, particularly in areas where patients generate an excess of saliva, mucous, or similar bodily fluids—or otherwise has difficult in draining such fluids. The wearable aspiration device is generally referred to by reference numeral 10. The wearable aspiration device 10 is useful for helping to drain excess saliva, mucous, or similar bodily fluids from a patient so as to minimize and/or reduce the risk of infection, sores, or other medical harm.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the,” are intended to include the plural forms as well as the singular forms, unless the context clearly indicates otherwise. It will be further understood that the terms “comprises” and/or “comprising,” when used in this specification, specify the presence of stated features, steps, operations, elements and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps and/or elements in an unnecessary fashion. Nevertheless, the specification and claims should be read with the understanding that such combinations are entirely within the scope of the invention and the claims.

The present specification herein describes a new wearable suction aspirator system for removing patient airway secretions in and around the nose, mouth, and/or tracheostomy tube. In the following description, for the purposes of explanation, numerous specific details are set forth in order to provide a thorough understanding of the present invention. It will be evident, however, to one skilled in the art that the present invention may be practiced without all of these specific details.

The present disclosure is to be considered as an exemplification of the invention and is not intended to limit the invention to the specific embodiments illustrated by the figures of the description below.

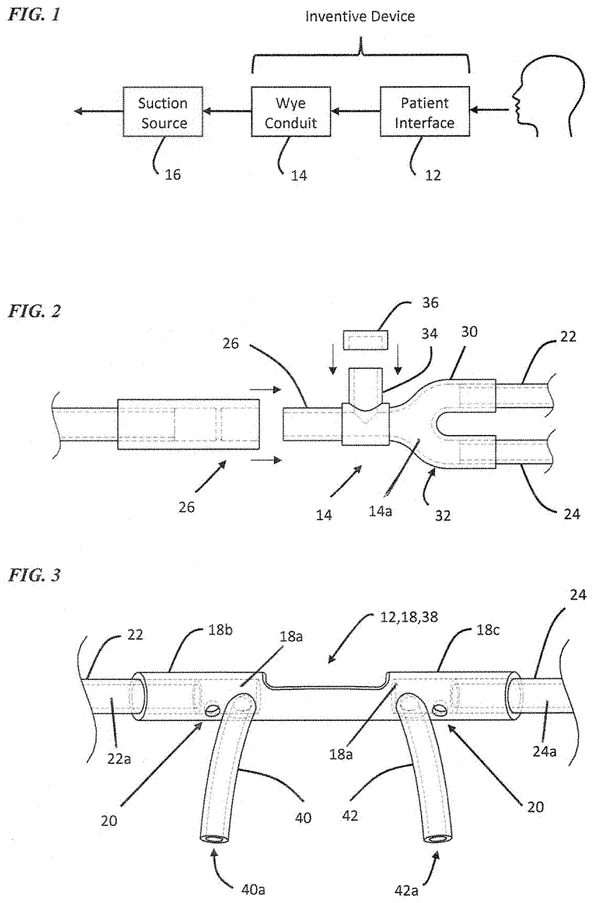

The present invention will now be described by referencing the appended figures representing preferred embodiments. FIG. 1 shows a general overview of how the system provides a negative pressure (suction) to the patient's oral or nasal passages, or around a tracheostomy tube. The negative pressure from the suction source draws secretions away and removes the secretions from the patient through openings in the patient interface on a continual or intermittent basis.

As shown in FIG. 1, the elements of the inventive system 10 include a patient interface 12, a wye conduit 14, and a suction source 16. FIG. 2 generally illustrates the wye conduit 14 and its connection to the suction source 16. FIGS. 3-5A illustrate various embodiments of the patient interface 12.

Across all embodiments, the patient interface 12 generally has a tubular body 18 and at least two aspiration openings 20. The aspiration openings 20 are in fluid communication with an internal passage 18a (shown in broken lines) on the inside of the tubular body. The interface 12 is configured to adapt to an oral, nasal, or tracheostomy opening, as described more fully below. First and second airway tubes 22,24 are each connected endwise to the tubular body 18. The airway tubes 22,24, both have respective internal passages 22a, 24a (shown in broken lines) that, when connected endwise to the ends 18b, 18c of the tubular body 18, are in fluid communication with the internal passage 18a of the tubular body 18. The internal passage 18a may pass completely through (not shown) the tubular body 18 so as to fluidly connect opposite ends 18b, 18c—or may pass partway through the body 18 so as to fluidly connect the ends 18b, 18c to the aspiration openings 20 or other structures as described below (FIGS. 3-5A).

FIG. 2 shows the wye conduit 14 connecting the negative pressure from suction source 16 to airway tubes 22 and 24. The wye conduit 14 has a conduit inlet 26 that is configured to connect to an external suction source or circuit 28. Opposite the conduit inlet 26, the wye conduit 14 has two conduit branches 30, 32, which are in turn connected endwise to each of the airway tubes 22, 24. The wye conduit 14 acts to separate the conduit inlet 26 into the two conduit branches 30 and 32. An internal passage 14a (shown in broken lines) permits fluid communication from the conduit inlet 26 to each of the conduit branches 30, 32. This same internal passage 14a, through the conduit branches 30, 32 is also in fluid communication with the internal passages 22a, 24a of the airway tubes 22, 24. Because of this fluid communication, the negative pressure downstream from suction source 26 causes a flow through wye conduit 14 creating suction on airway tubes 22 and 24.

The wye conduit 14 may also include a side access port 34 that is disposed in between the conduit inlet 26 and the conduit branches 30, 32. The side access port 34 provides fluid access to the internal passage 14a so as to permit the introduction of water, saline, or other cleaning fluid to clear the wye conduit 14. A removable cap 36 is friction-fit disposed on the side access port 34 to selectively open the port 34 when cleaning is necessary and close the port 34 when vacuum is necessary. The side access port 34 provides access for flushing the internal passage 14a with water, saline, other solvent.

In a first preferred embodiment of the patient interface 12, shown in FIG. 3, the patient interface 12 comprises an oral patient interface 38. The oral patient interface 38 has a tubular body 18 and at least two aspiration openings 20 as described above. The first and second airway tubes, 22, 24 are respectively attached to opposite ends 18b, 18c of the tubular body 18. In addition, first and second oral insert tubes 40, 42 are attached to the tubular body 18, preferably proximate to the aspiration openings 20. The first and second oral insert tubes 40, 42 have openings 40a, 42a respectively, that are in fluid communication with the internal passage 18a, which extends from the ends 18b, 18c to at least the point of the insert tubes 40, 42.

The oral insert tubes 40, 42 are configured for insertion into the patient's mouth and configured so as to aspirate bodily fluids, i.e., saliva, phlegm, etc., while the same are still inside the patient's mouth. In addition, the openings 20 being disposed proximate to opposite ends of the patient's mouth are configured to aspirate any excess bodily fluids that are not aspirated by the openings 40a, 42a and manage to make it out of the patient's mouth. The first and second oral insert tubes 40, 42 are configured to be disposed in either side of the patient's mouth.

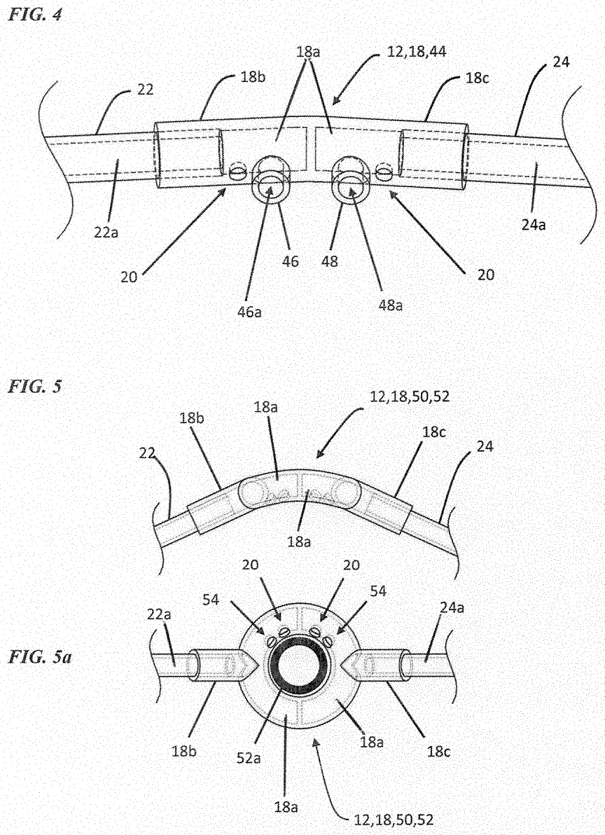

In a second preferred embodiment of the patient interface 12, shown in FIG. 4, the patient interface 12 comprises a nasal patient interface 44. The nasal patient interface 44 has a tubular body 18 and at least two aspiration openings 20 as described above. The first and second airway tubes, 22, 24 are respectively attached to opposite ends 18b, 18c of the tubular body 18. In addition, first and second nares tubes 46, 48 are attached to the tubular body 18, preferably proximate to the aspiration openings 20. The first and second nares tubes 46, 48 have openings 46a, 48a respectively, that are in fluid communication with the internal passage 18a, which extends from the ends 18b, 18c to at least the point of the nares tubes 46, 48.

The nares tubes 46, 48 are configured for minimal insertion into the entrance of the patient's nares and configured so as to aspirate bodily fluids, i.e., mucous, etc., while the same are still inside the patient's nose. In addition, the openings 20 being disposed proximate to opposite sides of the patient's nares are configured to aspirate any excess bodily fluids that are not aspirated by the openings 46a, 48a and manage to make it out of the patient's nose. While the first and second nares tubes 46, 48 are configured to be disposed in either of the patient's nares, such extend minimally into the patient's nares to avoid causing discomfort for the patient.

In a third preferred embodiment of the patient interface 12, shown in FIGS. 5 and 5A, the patient interface 12 comprises a tracheostomy patient interface 50. The tracheostomy patient interface 50 has a tubular body 18 and at least two aspiration openings 20 as described above. The tubular body 18 is preferably formed into a torus or donut shape 52, with a central opening 52a and ends 18b, 18c extending away from opposite sides of the torus shape 52. The torus or donut shape 52 is preferably formed with a slight curvature relative to a latitudinal plane of the torus shape 52. The first and second airway tubes, 22, 24 are respectively attached to opposite ends 18b, 18c of the tubular body 18. Additional aspiration openings 54 may be included in the tubular body 18, disposed around the central opening 52a along with the aspiration openings 20.

The central opening 52a is configured such that the torus or donut shape 52 is disposed around a tracheostomy tube 56. The slight curvature in the torus or donut shape 52 allows for the same to rest against and follow the natural curvature of the patient's neck. The aspiration openings 20, 54 are in fluid communication with the internal passage 18a, which extends from the ends 18b, 18c to at least the point of the aspiration openings 20, 54—so as to effectively split the torus/donut shape 52 in half. The aspiration openings 20, 54, in fluid communication with the internal passage 18a, are configured so as to aspirate bodily fluids, i.e., mucous, etc., that may ooze out from around the tracheostomy tube 56.

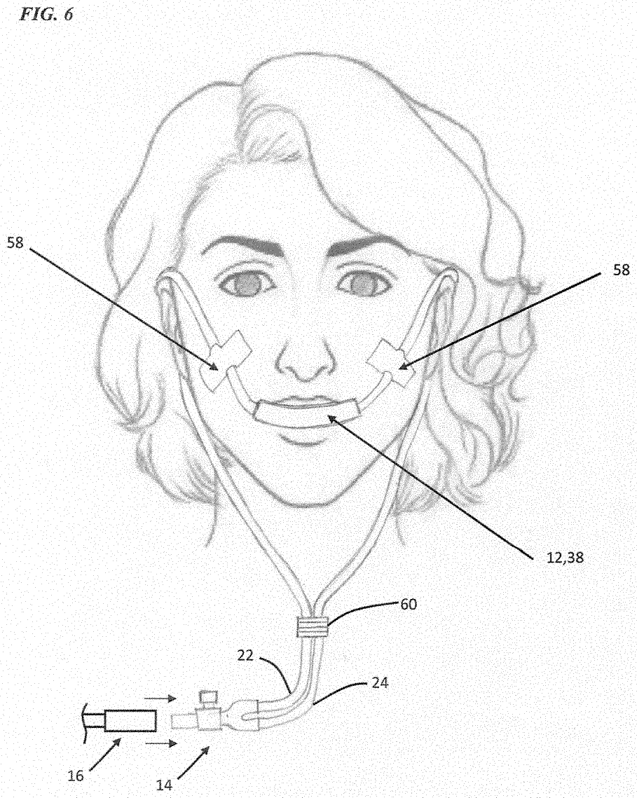

FIG. 6 shows use of the first preferred embodiment 38 of the wearable suction aspirator attached to a patient for use in oral suctioning. The suction circuit 16 is attached to wye conduit 14 which is connected to flexible tubes 22 and 24 which is connected to patient interface 38. The tubes 22 and 24 loop over the ears and are attached to the cheeks by tape 58 or similar retention devices. Tube tie 60 may be moved along tubes 22 and 24 and is used to adjust patient fit.

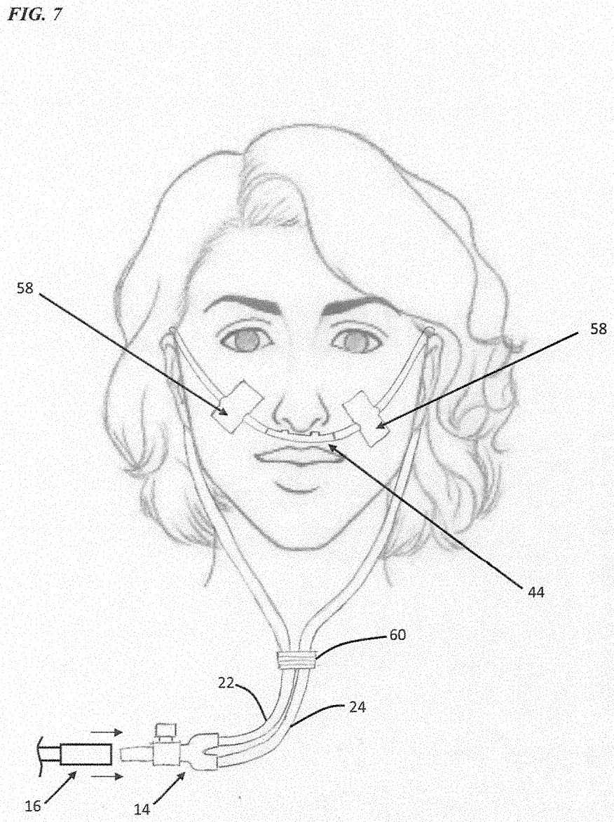

FIG. 7 shows use of the second preferred embodiment 44 of the wearable suction aspirator attached to a patient for use in nasal suctioning. The suction circuit 16 is attached to the wye conduit 14 which is connected to tubes 22 and 24 which is connected to patient interface 44. The tubes 22 and 24 loop over the ears and are attached to the cheeks by tape 58 or similar retention device. Tube tie 60 may be moved along tubes 22 and 24 and is used to adjust patient fit.

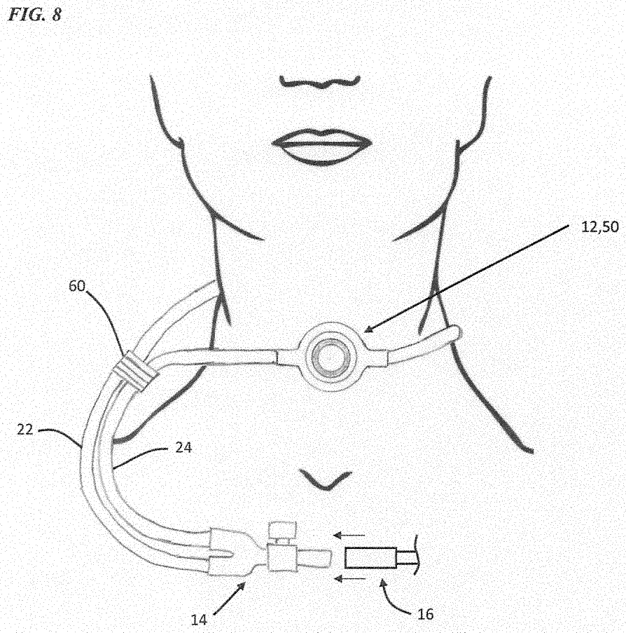

FIG. 8 shows use of the third preferred embodiment 50 of the wearable suction aspirator attached to a patient's tracheostomy tube 56 for suctioning. The suction circuit 16 is attached to wye conduit 14 which is connected to tubes 22 and 24 which is connected to patient interface 50. The tubing 22 and 24 loop around the neck of the patient and tube tie 60 may be moved along tubes 22 and 24 and is used to adjust patient fit.

The patient wearable suction aspirator interfaces 12, 38, 44, and 50 are each shown in their respective uses in FIGS. 6, 7, and 8. The interfaces 12, 28, 44, and 50 can be attached in other manners on or near the patient for use in nasal, oral and tracheostomy suctioning without departing from the spirit and scope of the invention. The patient interfaces 12, 28, 44, and 50 can be of variable sizes and lengths depending on the patient size.

Although several embodiments have been described in detail for purposes of illustration, various modifications may be made without departing from the scope and spirit of the invention. Accordingly, the invention is not to be limited, except as by the appended claims.

What is claimed is:

1. A wearable suction catheter, comprising:
a plastic patient aspirator interface having a tubular body and at least two aspiration openings;
a first airway tube and a second airway tube, both connected endwise to opposite ends of the tubular body; and
a wye conduit having a conduit inlet and first and second conduit branches, wherein the first conduit branch is connected to the first airway tube and the second conduit branch is connected to the second airway tube, and the conduit inlet is configured for connection to a vacuum source.

2. The wearable suction catheter of claim 1, wherein the tubular body is configured for attachment to a patient's mouth such that the at least two aspiration openings are disposed proximate to either side of the patient's mouth.

3. The wearable suction catheter of claim 2, the patient aspirator interface further having an oral aspiration tube configured for insertion into one side of the patient's mouth.

4. The wearable suction catheter of claim 3, the patient aspirator interface having two oral aspiration tubes configured for insertion into both sides of the patient's mouth.

5. The wearable suction catheter of claim 1, wherein the tubular body is configured for attachment to a patient's nares such that the at least two aspiration openings are disposed proximate to either side of the patient's nares.

6. The wearable suction catheter of claim 4, the patient aspirator interface further having two nasal aspiration tubes configured for insertion into the patient's nares.

7. The wearable suction catheter of claim 1, wherein the tubular body is shaped as a torus and configured for disposal around a patient's tracheostomy tube and has a latitudinal curvature configured to match a patient's neck proximate to the tracheostomy tube, such that the aspiration openings are disposed proximate to the tracheostomy tube.

8. The wearable suction catheter of claim 7, the patient aspirator interface having four aspiration openings.

9. The wearable suction catheter of claim 1, further comprising a cleaning port in the wye conduit disposed between the conduit inlet and the conduit branches.

10. The wearable suction catheter of claim 9, further comprising a cap on the cleaning port configured for selectively opening the cleaning port.

11. A wearable suction catheter, comprising:
a plastic patient aspirator interface having a tubular body and at least two aspiration openings, wherein the tubular body is configured for attachment to a patient's mouth or nares such that the at least two aspiration openings are disposed proximate to either side of the patient's mouth or nares;
a first airway tube and a second airway tube, both connected endwise to opposite ends of the tubular body; and
a wye conduit having a conduit inlet and first and second conduit branches, wherein the first conduit branch is connected to the first airway tube and the second conduit branch is connected to the second airway tube, and the conduit inlet is configured for connection to a vacuum source.

12. The wearable suction catheter of claim 11, the patient aspirator interface further having two oral aspiration tubes configured for insertion into both sides of the patient's mouth or two nasal aspiration tubes configured for insertion into the patient's nares.

13. The wearable suction catheter of claim 11, further comprising a cleaning port in the wye conduit disposed between the conduit inlet and the conduit branches.

14. The wearable suction catheter of claim 13, further comprising a cap on the cleaning port configured for selectively opening the cleaning port.

15. A wearable suction catheter, comprising:
a plastic patient aspirator interface having a tubular body and at least four aspiration openings, wherein the tubular body is shaped as a torus and configured for disposal around a patient's tracheostomy tube and has a latitudinal curvature configured to match a patient's neck proximate to the tracheostomy tube, such that the aspiration openings are disposed proximate to the tracheostomy tube;
a first airway tube and a second airway tube, both connected endwise to opposite ends of the tubular body; and
a wye conduit having a conduit inlet and first and second conduit branches, wherein the first conduit branch is connected to the first airway tube and the second conduit branch is connected to the second airway tube, and the conduit inlet is configured for connection to a vacuum source.

16. The wearable suction catheter of claim 15, further comprising a cleaning port in the wye conduit disposed between the conduit inlet and the conduit branches.

17. The wearable suction catheter of claim 16, further comprising a cap on the cleaning port configured for selectively opening the cleaning port.
